(19) United States
(12) Patent Application Publication
(10) Pub. No.: US 2026/0274796 A1
(43) Pub. Date: Sep. 17, 2026

(54) BIS-PYRIDINIUM COMPOUNDS, MATERIALS INCLUDING SUCH, AND USES THEREOF SUCH AS IN CORROSION RESISTANCE

(71) Applicant: Saudi Arabian Oil Company, Dhahran (SA)

(72) Inventors: Muhammad Imran Ul-Haq, Dhahran (SA); Qasim Saleem, Khobar (SA); Mram Alyami, Riyadh (SA)

(73) Assignee: Saudi Arabian Oil Company, Dhahran (SA)

(21) Appl. No.: 19/076,270

(22) Filed: Mar. 11, 2025

(51) Int. Cl. C07D 213/06; C09K 8/74; C23F 11/14

(52) U.S. Cl. CPC C07D 213/06; C09K 8/74; C23F 11/149; C09K 2208/32

(57) ABSTRACT

Provided are bis-pyridinium compounds, in which the two pyridinium groups are connected by a spacer through functionalized groups containing oxygen, nitrogen, and/or sulfur. Also provided are materials and formulations including such bis-pyridinium compounds. Also provided are corrosion-resistant substrates and films including such bis-pyridinium compounds. Also provided are processes for inhibiting corrosion of a metallic surface using the formulations and processes of acidizing a subsurface formation using the bis-pyridinium compounds.

### TECHNICAL FIELD

The present disclosure relates to chemical compounds and their uses and, more specifically, to chemical compounds that may exhibit corrosion-resistant functionality.

### BACKGROUND

Corrosion is an issue for many materials when they interact with their environments over time. For example, the presence of species such as H₂S, CO₂, organic acids, and brine solutions in produced oil may create a corrosive environment for transportation pipelines and oil processing units in an oil and gas facility. Specifically, when CO₂ and H₂S are dissolved in water, these species may behave like weak acids and promote the corrosion of steel, thus resulting in damage to the internal walls of the transportation pipelines and oil processing units and causing leaks that will increase the maintenance time and costs associated with the oil and gas processing. Many conventional compounds may be used in corrosion inhibitors and corrosion-resistant films in order to reduce corrosion of surfaces. However, these conventional compounds are often toxic and non-biodegradable. Additionally, there is a relatively high cost associated with the production of these conventional compounds. Further, these conventional compounds do not sufficiently resist the corrosive effects present in the wet sour environment (i.e., an environment rich in H₂S, e.g., greater than 0.5 wt. %) often present in crude oil processing facilities. As such, new compounds are needed for use in corrosion inhibitors and corrosion-resistant films.

### BRIEF SUMMARY

Described herein are bis-pyridinium compounds that, in some embodiments, may be useful for inhibiting corrosion, as well as materials that include these bis-pyridinium compounds. Such bis-pyridinium compounds include two connected pyridinium functional groups. The two pyridinium functional groups, where the nitrogen atom of each pyridinium functional group is further functionalized by including at least one additional heteroatom chosen from nitrogen, oxygen, and sulfur, as described herein, are connected and spaced by an alkyl chain of varying numbers (n) of carbon atoms. The bis-pyridinium compounds described herein may reduce corrosion of metallic surfaces, such as carbon steel. For example, and without limitation, these bis-pyridinium compounds may be present in well fluids, formulations comprising water or alcoholic solvents, and corrosion-resistant substrates, particularly where in contact with sour environments that include elemental sulfur or sulfur-containing compounds such as hydrogen sulfide. Additionally, these bis-pyridinium compounds may be utilized in methods for inhibiting corrosion by contact with a substrate such as a pipe, or in acidizing processes. The presence of bis-pyridinium compounds may, in some embodiments, perform as well or better than conventional compounds known in inhibit corrosion.

According to one or more embodiments of the present disclosure, a bis-pyridinium compound may have the structure of Chemical Structure #1, as is described herein, or a salt thereof. In Chemical Structure #1, X¹ and X² may be each independently chosen from —CH₃, —NH₂, —OH, —SH, or —H. Y¹ and Y² may be each independently chosen from —CH₂—, —NH—, —O—, or —S—. X¹ and X² are not —H when Y¹ and Y² are —CH₂—. The two pyridinium groups may be connected by an alkyl chain of varying numbers (n) of carbon atoms, where n may by any integer greater than or equal to zero (0). The two pyridinium groups may include R^{A1}, R^{B1}, R^{C1}, R^{D1}, R^{E1}, R^{A2}, R^{B2}, R^{C2}, R^{D2}, and R^{E2} that are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

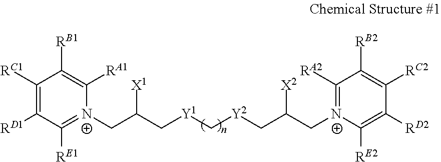

According to one or more additional embodiments of the present disclosure, a material may comprise a bis-pyridinium compound. The bis-pyridinium compound may have the structure of Chemical Structure #1, as is described herein, or a salt thereof. In Chemical Structure #1, X¹ and X² may be each independently chosen from —CH₃, —NH₂, —OH, —SH, or —H. Y¹ and Y² may be each independently chosen from —CH₂—, —NH—, —O—, or —S—. X¹ and X² are not —H when Y¹ and Y² are —CH₂—. The two pyridinium groups may be connected by an alkyl chain of varying numbers (n) of carbon atoms, where n may by any integer greater than or equal to zero (0). The two pyridinium groups may independently include R^{A1}, R^{B1}, R^{C1}, R^{D1}, R^{E1}, R^{A2}, R^{B2}, R^{C2}, R^{D2}, and R^{E2} that are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

According to one or more yet additional embodiments of the present disclosure, a corrosion-resistant substrate may comprise a substrate comprising a first surface and a corrosion-resistant film positioned on at least a portion of the first surface of the substrate. The corrosion-resistant film may comprise a bis-pyridinium compound. The bis-pyridinium compound may have the structure of Chemical Structure #1, as is described herein, or a salt thereof. In Chemical Structure #1, X¹ and X² may be each independently chosen from —CH₃, —NH₂, —OH, —SH, or —H. Y¹ and Y² may be each independently chosen from —CH₂—, —NH—, —O—, or —S—. X¹ and X² are not —H when Y¹ and Y² are —CH₂—. The two pyridinium groups may be connected by an alkyl chain of varying numbers (n) of carbon atoms, where n may by any integer greater than or equal to zero (0). The two pyridinium groups may independently include R^{A1}, R^{B1}, R^{C1}, R^{D1}, R^{E1}, R^{A2}, R^{B2}, R^{C2}, R^{D2}, and R^{E2} that are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

According to one or more yet additional embodiments of the present disclosure, a subsurface formation may be acidized by a method that may comprise injecting an acidic treatment fluid into an subsurface formation through a tubing of an extraction well, thereby exposing at least a portion of the tubing to an acidic environment. At least a portion of the tubing may contacted by a bis-pyridinium compound. The bis-pyridinium compound may have the structure of Chemical Structure #1, as is described herein, or a salt thereof. In Chemical Structure #1, X¹ and X² may be each independently chosen from —CH₃, —NH₂, —OH, —SH, or —H. Y¹ and Y² may be each independently chosen from —CH₂—, —NH—, —O—, or —S—. X¹ and X² are not —H when Y¹ and Y² are —CH₂—. The two pyridinium groups may be connected by an alkyl chain of varying numbers (n) of carbon atoms, where n may by any integer greater than or equal to zero (0). The two pyridinium groups may independently include R^{A1}, R^{B1}, R^{C1}, R^{D1}, R^{E1}, R^{A2}, R^{B2}, R^{C2}, R^{D2}, and R^{E2} that are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

These and other embodiments are described in more detail in the detailed description. It is to be understood that both the foregoing general description and the following detailed description present embodiments of the presently disclosed technology, and are intended to provide an overview or framework for understanding the nature and character of the presently disclosed technology as it is claimed. The accompanying drawings are included to provide a further understanding of the presently disclosed technology and are incorporated into and constitute a part of this specification. The drawings illustrate various embodiments and, together with the description, serve to explain the principles and operations of the presently disclosed technology. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and wherein:

FIG. 1 schematically depicts a cross-sectional view of a substrate comprising a corrosion-resistant film, according to one or more embodiments shown and described herein;

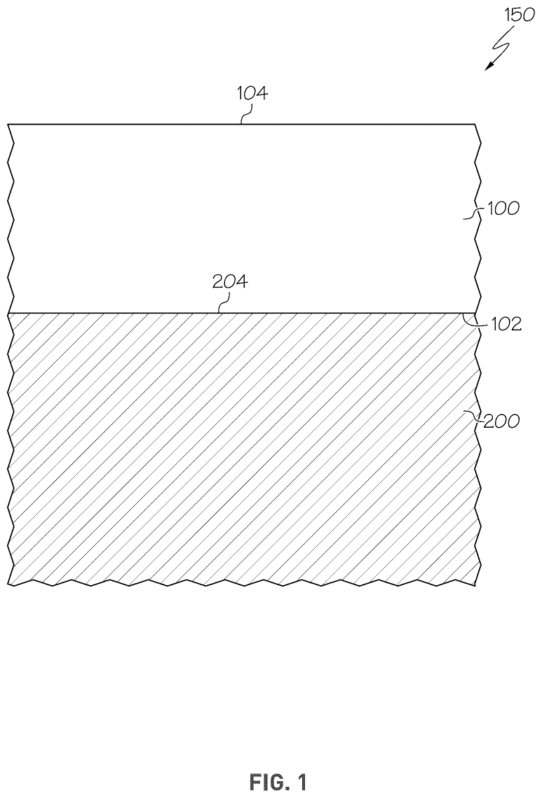

FIG. 2 schematically depicts a cross-sectional view cut in the axial direction of a metal pipe comprising a corrosion-resistant film, according to one or more embodiments shown and described herein;

FIG. 3 schematically depicts a hydrocarbon extraction well, according to one or more embodiments shown and described herein;

FIG. 4 graphically depicts the ¹H-NMR spectrum of a synthesized bis-pyridinium compound of Chemical Structure #2-1, according to one or more embodiments shown and described herein;

FIG. 5 graphically depicts the ¹³C {¹H}-NMR spectrum of the synthesized bis-pyridinium compound of FIG. 4, according to one or more embodiments shown and described herein;

FIG. 6 graphically depicts the mass spectrum of a synthesized bis-pyridinium compound of FIG. 4, according to one or more embodiments shown and described herein; and

FIG. 7 graphically depicts the ¹H-NMR spectrum of a synthesized bis-pyridinium compound of Chemical Structure #3-1, according to one or more embodiments shown and described herein.

### DETAILED DESCRIPTION

One or more embodiments of the present disclosure are directed to bis-pyridinium compounds that are capable of inhibiting corrosion. The bis-pyridinium compounds described herein may generally be depicted by Chemical Structure #1. As shown in Chemical Structure #1, the bis-pyridinium compounds have two connected pyridinium groups. The two pyridinium groups are independently functionalized by X¹ and Y¹ or X² and Y². X¹ and X² are each independently chosen from —CH₃, —NH₂, —OH, —SH, or —H; and Y¹ and Y² are each independently chosen from —CH₂—, —NH—, —O—, or —S—. However, X¹ and X² are not —H when Y¹ and Y² are —CH₂—.

According to some embodiments, the bis-pyridinium compounds may include two charged cations, as shown in Chemical Structure #1, where the N atom of each pyridinium unit is shown as having a positive charge. In additional embodiments, the bis-pyridinium compounds may be salts, where two anions (not shown) are bonded with the bis-pyridinium compounds of Chemical Structure #1. For example, and without limitation, anions may include F—, Cl—, Br, or I—. Non-limiting examples of salts include chloride and bromide compounds of Chemical Structure #1.

In embodiments, if X¹ and X² are —OH, Y¹ and Y² may each independently be chosen from —CH₂—, —NH—, —O—, or —S—. Non-limiting examples include bis-pyridinium compounds in which X¹ and X² are —OH and Y¹ and Y² are —CH₂—, bis-pyridinium compounds in which X¹ and X² are —OH and Y¹ and Y² are —O—, bis-pyridinium compounds in which X¹ and X² are —OH and Y¹ and Y² are —S—, and bis-pyridinium compounds in which X¹ and X² are —OH and Y¹ and Y² are —NH—.

In other embodiments, if X¹ and X² are —NH₂, Y¹ and Y² may each independently be chosen from —CH₂—, —NH—, —O—, or —S—. Non-limiting examples include bis-pyridinium compounds in which X¹ and X² are —NH₂ and Y¹ and Y² are —NH—, bis-pyridinium compounds in which X¹ and X² are —NH₂ and Y¹ and Y² are —O—, bis-pyridinium compounds in which X¹ and X² are —NH₂ and Y¹ and Y² are —S—, and bis-pyridinium compounds in which X¹ and X² are —NH₂ and Y¹ and Y² are —CH₂—.

In other embodiments, if X¹ and X² are —SH, Y¹ and Y² may each independently be chosen from —CH₂—, —NH—, —O—, or —S—. Non-limiting examples include bis-pyridinium compounds in which X¹ and X² are —SH and Y¹ and Y² are —S—, bis-pyridinium compounds in which X¹ and X² are —SH and Y¹ and Y² are —NH—, and bis-pyridinium compounds in which X¹ and X² are —SH and Y¹ and Y² are —O—, and bis-pyridinium compounds in which X¹ and X² are —SH and Y¹ and Y² are —CH₂—.

In additional embodiments, if X¹ and X² are —H or X¹ and X² are —CH₃, Y¹ and Y² may each independently be chosen from —NH—, —O—, or —S—. Non-limiting examples include bis-pyridinium compounds in which X¹ and X² are —H or X¹ and X² are —CH₃ and Y¹ and Y² are —NH—, bis-pyridinium compounds in which X¹ and X² are —H or X¹ and X² are —CH₃ and Y¹ and Y² are —O—, and bis-pyridinium compounds in which X¹ and X² are —H or X¹ and X² are —CH₃ and Y¹ and Y² are —S—,

Without being bound by any particular theory, it is believe that, compared to mono-pyridinium compounds, bis-pyridinium compounds exhibit low foaming tendency, a greater number of binding sites, and high thermal stability. Moreover, bis-pyridinium compounds that are functionalized by having nitrogen or sulfur as X¹, X², Y¹, or Y² could provide additional binding sites for adsorption (e.g. physisorption and/or chemisorption) on the metal surface and yield high corrosion protection. Furthermore, bis-pyridinium compounds provide additional capability to adjust the distance between the two pyridinium moieties to balance the foaming tendency and surface protection. As illustrated herein, the nitrogen atoms of the two pyridinium moieties are connected through a chain of atoms that include a spacer having n atoms connected between two functionalized groups independently having X (e.g. X¹ or X²) chosen from —CH₃, —NH₂, —OH, —SH, or —H and/or Y (e.g. Y¹ or Y²) chosen from —CH₂—, —NH—, —O—, or —S—. In embodiments, the nitrogen atoms of the two pyridinium moieties may be connected through a chain of atoms that have a total number of atoms (Nₜₒₜₐₗ) from 6 to 16, from 8 to 16, from 12 to 16, from 6 to 12, or from 8 to 12, determined based on the atoms directly or indirectly connected between the nitrogen atoms of both pyridinium moieties. For example, and as shown in chemical structures illustrated herein, a bis-pyridinium compound having the structure of Chemical Structure #1 and n=0 has a total number of atoms, Nₜₒₜₐₗ, that is equal to 8, and a bis-pyridinium compound having the structure of Chemical Structure #1 and n=4 has the total number of atoms, Nₜₒₜₐₗ, that is equal to 12. Depending on the selection of the functionalized groups, and as exemplified in Chemical Structure #1, the spacer connecting the two functional groups may be an alkyl chain. The alkyl chain spacer may have a number (n) of carbon atoms. The numbers of carbon atoms (n) may by any integer greater than or equal to zero (0), such as from 0 to 8, from 0 to 6, from 0 to 4, from 0 to 2, from 2 to 8, from 2 to 6, from 2 to 4, from 4 to 8, from 4 to 6, or from 6 to 8. In some embodiments, n may be 0 or 4.

In embodiments, when n is zero, Y¹ and Y² are directly connected as shown in Chemical Structure #2. Non-limiting examples include bis-pyridinium compounds in which Y¹ and Y² are —CH₂—, X¹ and X² are independently chosen from —NH₂, —OH, or —SH, and n is zero; bis-pyridinium compounds in which Y¹ and Y² are —CH₂—, X¹ and X² are —OH, and n is zero; and bis-pyridinium compounds in which Y¹ and Y² are CH₂—, X¹ and X² are —SH, and n is zero. The bis-pyridinium compounds in which Y¹ and Y² are —CH₂—, X¹ and X² are —OH, and n is zero are depicted in Chemical Structure #2-1. The bis-pyridinium compounds in which Y¹ and Y² are —CH₂—, X¹ and X² are —SH, and n is zero are depicted in Chemical Structure #2-2.

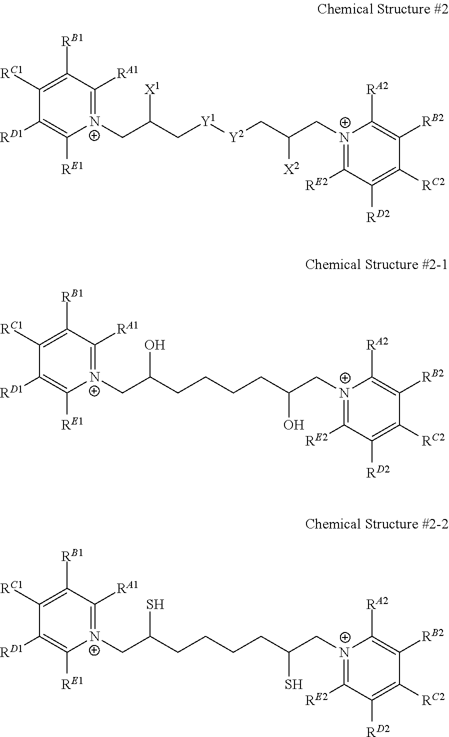

In other embodiments, when n is greater than zero, (e.g. n equals to 1 or 4), Y¹ and Y² are connected as shown in Chemical Structure #3. Non-limiting examples include bis-pyridinium compounds in which Y¹ and Y² are —O—, X¹ and X² are independently chosen from —NH₂, —OH, —SH, —CH₃, or —H, and n is four; bis-pyridinium compounds in which Y¹ and Y² are —O—, X¹ and X² are —OH, and n is four; bis-pyridinium compounds in which Y¹ and Y² are —CH₂—, X¹ and X² are independently chosen from —NH₂, —OH, —SH, —CH₃, or —H, and n is one; and bis-pyridinium compounds in which Y¹ and Y² are —CH₂—, X¹ and X² are independently chosen from —NH₂, and n is one. The bis-pyridinium compounds in which Y¹ and Y² are —O—, X¹ and X² are —OH, and n is four are depicted in Chemical Structure #3-1. The bis-pyridinium compounds in which Y¹ and Y² are —CH₂—, X¹ and X² are —NH₂, and n is one are depicted in Chemical Structure #3-2.

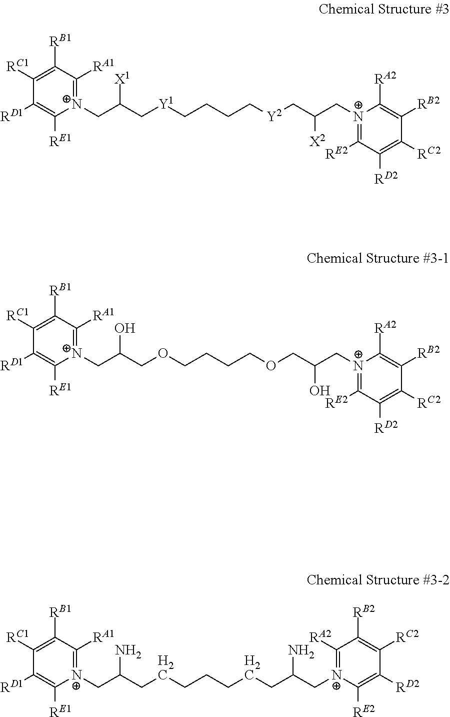

Referring to Chemical Structure #1, as well as other chemical structures describe herein, the general structure includes R^{A1}, R^{B1}, R^{C1}, R^{D1}, R^{E1}, R^{A2}, R^{B2}, R^{C2}, R^{D2}, and R^{E2} that each represent various functional groups. Unless specified otherwise, discussion herein of R groups may refer to the disclosed bis-pyridinium compounds represented by Chemical Structure #1. R^{A1}, R^{B1}, R^{C1}, R^{D1}, R^{E1}, R^{A2}, R^{B2}, R^{C2}, R^{D2}, and R^{E2} may each independently be chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group. Without being bound by a theory, it is believed that one or more of R^{A1}, R^{B1}, R^{C1}, R^{D1}, R^{E1}, R^{A2}, R^{B2}, R^{C2}, R^{D2}, and R^{E2} groups having a relatively long carbon chain moiety allow the bis-pyridinium compound to better adhere to a metal surface, such as tubing. Further, if the carbon chain moiety has greater than 18 carbon atoms, there may be an increased risk of the bis-pyridinium compound having poor adherence to metal substrates.

In one or more embodiments, the term “functional group” or “group” may refer to a substituent or moiety that is present in the bis-pyridinium compound. For example, when the disclosure states that R^{C1} may be a methyl group, the methyl group (—CH₃) replaces R^{C1} of the general structure of the bis-pyridinium compounds disclosed and described herein, where the carbon atom of the methyl group is now bonded to the C atom of pyridinium unit to which R^{C1} bonded.

As described herein, moieties may be defined by the number of carbon atoms included in the moiety, such as Cₓ-C_{y}, where x is the least number of carbon atoms and y is the greatest number of carbon atoms contemplated. For example, C₁-C₁₈ describes a moiety that has from 1 to 18 carbon atoms.

In some embodiments, R^{A1}, R^{B1}, R^{C1}, R^{D1}, R^{E1}, R^{A2}, R^{B2}, R^{C2}, R^{D2}, and R^{E2} may each independently be a C₁-C₁₈ alkyl group. The term “alkyl group” refers to a functional group that only contains carbon and hydrogen atoms, where the carbon atoms and hydrogen atoms are only connected by single bonds. In some embodiments, R^{A1}, R^{B1}, R^{C1}, R^{D1}, R^{E1}, R^{A2}, R^{B2}, R^{C2}, R^{D2}, and R^{E2} may each independently be a straight chained alkyl group having the chemical formula —(CH₂)ₓCH₃, where x is from 0 to 17, such as 0 (a methyl group), 1, 2, 3, 4, 5, 6, 7, 8, 9, 10, 11, 12, 13, 14, 15, 16, 17, or 18 carbon atoms. In additional embodiments, R^{A1}, R^{B1}, R^{C1}, R^{D1}, R^{E1}, R^{A2}, R^{B2}, R^{C2}, R^{D2}, and R^{E2} may each independently be branched alkyl groups having from 3 to 18 carbon atoms, such as 3, 4, 5, 6, 7, 8, 9, 10, 11, 12, 13, 14, 15, 16, 17, or 18 carbon atoms. In some embodiments, the alkyl group may include a ring structure, such as a pentane ring, a hexane ring, etc. For example, R^{A1}, R^{B1}, R^{C1}, R^{D1}, R^{E1}, R^{A2}, R^{B2}, R^{C2}, R^{D2}, and R^{E2} may each be methyl, ethyl, decyl (i.e., a C₁₀ alkyl group), dodecyl (i.e., a C₁₂ alkyl group), tetraecyl, hexadecyl, or octadecyl.

In some embodiments, R^{A1}, R^{B1}, R^{C1}, R^{D1}, R^{E1}, R^{A2}, R^{B2}, R^{C2}, R^{D2}, and R^{E2} may each independently comprise a C₁-C₁₈ hydroxyl alkyl group. The term “hydroxyl alkyl group” refers to a functional group that includes one or more a hydroxyl moieties (—OH) bonded to an alkyl group. According to embodiments, the hydroxyl alkyl group may include 1, 2, 3, 4, 5, or even more hydroxyl moieties. In some embodiments, R^{A1}, R^{B1}, R^{C1}, R^{D1}, R^{E1}, R^{A2}, R^{B2}, R^{C2}, R^{D2}, and R^{E2} may each independently be a straight chained hydroxyl alkyl group having the chemical formula —(CH₂)ₓOH, where x is from 1 to 18. In additional embodiments, R^{A1}, R^{B1}, R^{C1}, R^{D1}R^{E1}, R^{A2}, R^{B2}, R^{C2}, R^{D2}, and R^{E2} may each independently be branched hydroxyl alkyl groups having from 1 to 18 carbon atoms and at least one hydroxyl group. For example, R^{A1}, R^{B1}, R^{C1}, R^{D1}, R^{E1}, R^{A2}, R^{B2}, R^{C2}, R^{D2}, and R^{E2} may each be ethyl alcohol.

In some embodiments, R^{A1}, R^{B1}, R^{C1}, R^{D1}, R^{E1}, R^{A2}, R^{B2}, R^{C2}, R^{D2}, and R^{E2} may each independently comprise a C₁-C₁₈ alkenyl group. The term “alkenyl group” refers to a functional group consisting of hydrogen and carbon atoms where at least two carbon atoms have a double bond. In some embodiments, the alkenyl group may have a single carbon to carbon double bond that is at the end of moiety (i.e., having the structure —(CH₂)ₓCH═CH₂, where x is from 0 to 16, such as 0, 1, 2, 3, 4, 5, 6, 7, 8, 9, 10, 11, 12, 13, 14, 15, or 16). For example, R^{A1}, R^{B1}, R^{C1}, R^{D1}, R^{E1}, R^{A2}, R^{B2}, R^{C2}, R^{D2}, and R^{E2} may each be propenyl.

In some embodiments, R^{A1}, R^{B1}, R^{C1}, R^{D1}, R^{E1}, R^{A2}, R^{B2}, R^{C2}, R^{D2}, and R^{E2} may each independently comprise a C₁-C₁₈ alkynyl group. The term “alkynyl group” refers to a functional group consisting of hydrogen and carbon atoms where at least two carbon atoms have a triple bond. In some embodiments, R^{A1}, R^{B1}, R^{C1}, R^{D1}, R^{E1}, R^{A2}, R^{B2}, R^{C2}, R^{D2}, and R^{E2} may each independently have a single carbon to carbon triple bond that is at the end of moiety (i.e., having the structure —(CH₂)ₓC═CH, where x is from 0 to 16, such as 0, 1, 2, 3, 4, 5, 6, 7, 8, 9, 10, 11, 12, 13, 14, 15, or 16). For example, R^{A1}, R^{B1}, R^{C1}, R^{D1}, R^{E1}, R^{A2}, R^{B2}, R^{C2}, R^{D2}, and R^{E2} may each be ethynyl or propynyl.

In some embodiments, R^{A1}, R^{B1}, R^{C1}, R^{D1}, R^{E1}, R^{A2}, R^{B2}, R^{C2}, R^{D2}, and R^{E2} may each independently comprise one or more of a carbon-carbon double bond, a carbon-carbon triple bond, or a combination thereof.

In some embodiments, R^{A1}, R^{B1}, R^{C1}, R^{D1}, R^{E1}, R^{A2}, R^{B2}, R^{C2}, R^{D2}, and R^{E2} may each independently be a C₁-C₁₈ acryl group. The term “acryl group” refers to a functional group consisting of a carbon-carbon double bond and a carbon-oxygen double bond separated by a carbon-carbon single bond. The acryl group may have the general formula —(CH₂)ₓCOCHCH₂, where x is any integer from 0 to 15, such as 0, 1, 2, 3, 4, 5, 6, 7, 8, 9, 10, 11, 12, 13, 14, or 15.

In some embodiments, R^{A1}, R^{B1}, R^{C1}, R^{D1}, R^{E1}, R^{A2}, R^{B2}, R^{C2}, R^{D2}, and R^{E2} may each independently be a C₁-C₁₈ functional alkyl group. The term “functional alkyl group” refers to an alkyl group that includes at least one moiety bonded to any carbon atom of the alkyl group. In some embodiments, the functional alkyl group may comprise more than one of the same moiety. In some embodiments, the functional alkyl group may comprise two or more different moieties. In some embodiments, the functional alkyl group may comprise a moiety chosen form a carboxyl group (i.e., —COOH), an amine group (i.e., —NH₂), or a thiol group (i.e., —SH).

In some embodiments, R^{A1}, R^{B1}, R^{C1}, R^{D1}, R^{E1}, R^{A2}, R^{B2}, R^{C2}, R^{D2}, and R^{E2} are each hydrogen. For example, the bis-pyridinium compounds disclosed herein may have X¹ and X² that are —OH, Y¹ and Y² that are —CH₂—, n that equals to zero, and R^{A1}, R^{B1}, R^{C1}, R^{D1}, R^{E1}, R^{A2}, R^{B2}, R^{C2}, R^{D2}, and R^{E2} that are each hydrogen. For example, the bis-pyridinium compounds disclosed herein may have X¹ and X² that are —OH, Y¹ and Y² that are —O—, n that equals to four, and R^{A1}, R^{B1}, R^{C1}, R^{D1}, R^{E1}, R^{A2}, R^{B2}, R^{C2}, R^{D2}, and R^{E2} that are each hydrogen.

As described herein, the spacer length (n) and the chain lengths of R^{A1}, R^{B1}, R^{C1}, R^{D1}, R^{E1}, R^{A2}, R^{B2}, R^{C2}, R^{D2}, and R^{E2} groups may influence the performance of the bis-pyridinium compounds. Thus, in some embodiments, the average molecular mass ranges (m/z) may be from 100 to 1500, such as from 100 to 1000, 100 to 500, 500 to 1500, or 500 to 1000. That is, for the doubly-charged bis-pyridinium compounds described and disclosed herein, the molecular mass may be from 200 to 3000, such as from 200 to 2000, from 200 to 1500, from 200 to 1000, from 200 to 500, from 500 to 3000, from 500 to 2000, from 500 to 1500, from 500 to 1000, from 1000 to 3000, from 1000 to 2000, from 1000 to 1500, from 1500 to 3000, from 1500 to 2000, or from 2000 to 3000.

According to one or more embodiments, the bis-pyridinium compounds described herein may be synthesized by a nucleophilic ring-opening reaction in an acidic environment, as is depicted in Reaction Scheme #1.

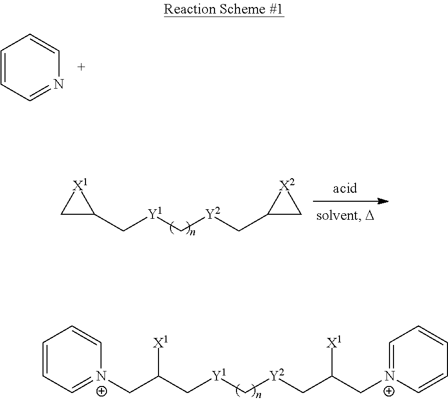

In Reaction Scheme #1, the long pair of electrons on the nitrogen atom of pyridine molecule attack and open the highly strained three-membered ring molecule, resulting in a functionalized bis-pyridinium compounds, as are depicted in Chemical Structures #1-3. The three-membered ring molecule may be chosen from an epoxy compound, an aziridine compound, a thiirane compound, or a cyclopropane compound.

In embodiments, the bis-pyridinium compounds in which X¹ and X² are —OH may be synthesized by reacting pyridine with an epoxy compound. For example, the epoxy compound may be a diepoxy alkane or a diglycidyl ether, such as 1,2,7,8-diepoxyoctane or 1,4-butanediol diglycidyl ether. The synthesis of example bis-pyridinium compounds in which X¹ and X² are —OH are subsequently described in Examples.

In embodiments, the bis-pyridinium compounds in which X¹ and X² are —NH₂ may be synthesized by reacting pyridines with aziridine compounds. For example, the aziridine compounds may be aziridinylalkyl aziridines. Reaction Scheme #2 illustrates the synthesis of a bis-pyridinium compound in which X¹ and X² are —NH₂, Y¹ and Y² are —CH₂—, n is one, and R^{A1}, R^{B1}, R^{C1}, R^{D1}, R^{E1}, R^{A2}, R^{B2}, R^{C2}, R^{D2}, and R^{E2} are each hydrogen.

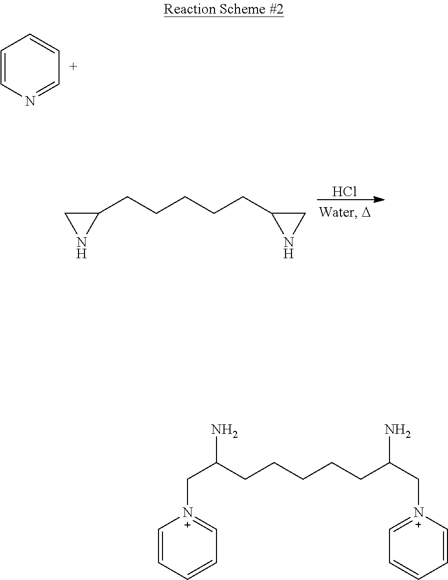 [SMILES: c1ccncc1 ; Cl ; C(CCC1CN1)CCC1CN1 ; NC(CCCCCC(N)C[n+]1ccccc1)C[n+]1ccccc1]

In Reaction Scheme #2, solutions of pyridine (2 mol), 2-[5-(Aziridin-2-yl) pentyl]aziridine (1 mol), and HCl (37%) (2 mol) in a closed vessel may be heated to a temperature between 65° C. to 110° C., such as at 90° C., for 24 h. After elapsed time, the reaction product is repeatedly washed with diethyl and freeze-dried to remove the water. The final product may be recrystallized in an alcohol mixture containing methanol and ethanol in a 1:1 ratio to obtain a solid bis-pyridinium compound. The synthesis reaction and the final compound are shown in Reaction Scheme #2 above.

In embodiments, the bis-pyridinium compounds in which X¹ and X² are —SH may be synthesized by reacting pyridines with thiirane compounds. For example, the thiirane compounds may be bis-thiiranes. Reaction Scheme #3 illustrates the synthesis of a bis-pyridinium compound in which X¹ and X² are —SH, Y¹ and Y² are —CH₂—, n is zero, and R^{A1}, RBI, R^{C1}, R^{D1}, R^{E1}, R^{A2}R^{B2}, R^{C2}, R^{D2}, and R^{E2} are each hydrogen.

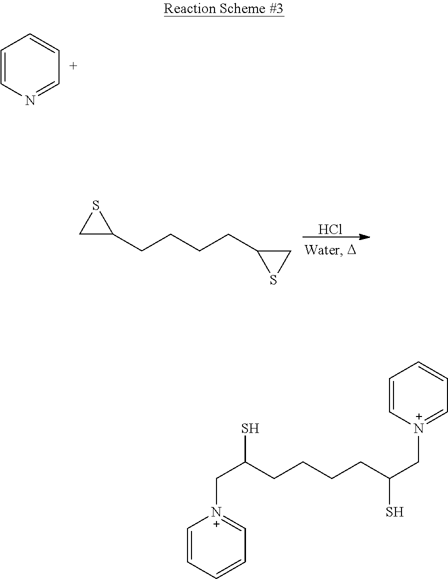 [SMILES: c1ccncc1 ; C(CCC1CS1)CC1CS1 ; Cl ; SC(CCCCC(S)C[n+]1ccccc1)C[n+]1ccccc1]

In Reaction Scheme #3, solutions of pyridine (2 mol), 2,2′-(1,4-Butanediyl)bis [thiirane](1 mol), and HCl (37%) (2 mol) in a closed vessel may be heated to a temperature between 65° C. to 110° C., such as at 90° C., for 24 h. After elapsed time, the reaction product is repeatedly washed with diethyl and freeze-dried to remove the water. The final product may be recrystallized in an alcohol mixture containing methanol and ethanol in a 1:1 ratio to obtain a solid bis-pyridinium compound. The synthesis reaction and the final compound are shown in Reaction Scheme #3 above.

According to additional embodiments, any one or a mixture of the bis-pyridinium compounds described herein may be utilized in a method for preventing corrosion and/or may be present in a material that may be utilized for preventing corrosion. In any of these materials and methods, one species or a combination of species of the bis-pyridinium compounds may be utilized. The described materials and/or methods may include any bis-pyridinium compound described herein, including without limitation those of Chemical Structures #1-#3.

In one or more embodiments, the bis-pyridinium compounds may be used to mitigate corrosion of a material. In some embodiments, the bis-pyridinium compounds may be used to mitigate corrosion of a metallic surface, such as a metallic surface in a wet sour environment. According to one or more embodiments, the bis-pyridinium compounds may be included in a well fluid to mitigate the corrosion of surfaces exposed to the well fluid, which may be acidic. Thus, a well fluid may include the bis-pyridinium compounds at a concentration by weight of from 0.1 parts per million to 100 parts per million, from 0.2 parts per million to 50 parts per million, from 0.2 parts per million to 40 parts per million, from 0.2 parts per million to 30 parts per million, from 0.2 parts per million to 20 parts per million, from 0.2 parts per million to 10 parts per million, from 0.3 parts per million to 8 parts per million, from 0.4 parts per million to 6 parts per million, from 0.5 parts per million to 5 parts per million, from 0.5 parts per million to 2 parts per million, or from 1 part per million to 2 parts per million of the well fluid. In embodiments, the well fluid may further comprise petroleum hydrocarbons.

In embodiments, a process for mitigating corrosion of a metallic surface may include contacting the metallic surface with a solution comprising the bis-pyridinium compounds at a concentration by weight of from 0.1 parts per million to 100 parts per million, from 0.2 parts per million to 50 parts per million, from 0.2 parts per million to 40 parts per million, from 0.2 parts per million to 30 parts per million, from 0.2 parts per million to 20 parts per million, from 0.2 parts per million to 10 parts per million, from 0.3 parts per million to 8 parts per million, from 0.4 parts per million to 6 parts per million, from 0.5 parts per million to 5 parts per million, from 0.5 parts per million to 2 parts per million, or from 1 part per million to 2 parts per million of the well fluid. According to embodiments, the metallic surface comprises steel. In some embodiments, the metallic surface comprises carbon steel. In embodiments, the metallic surface may be contacted with one or more bis-pyridinium compounds at least one time, at least two times, at least three times, at least four times, or even at least five times. It should be understood that when the metallic surface is contacted with the one or more bis-pyridinium compounds multiple times, each contacting may be accomplished using the same or different concentrations of the compound described herein.

In some embodiments, the one or more bis-pyridinium compounds described herein may be used as an active component in a formulation for inhibiting corrosion. According to one or more embodiments, a corrosion inhibitor formulation comprises the one or more bis-pyridinium compounds and water, or alcoholic solvents, or combinations thereof. In some embodiments, the corrosion inhibitor formulation further comprises a synergist, a surfactant, a supporting component, a secondary solvent, a coupling agent, an ethoxylated amine, or a combination thereof.

According to one or more embodiments, the one or more bis-pyridinium compounds may be present in the corrosion inhibitor formulation at a concentration of from 0.1 to 70 weight percent, from 0.1 to 60 weight percent, from 0.1 to 50 weight percent, from 0.1 to 40 weight percent, from 0.1 to 30 weight percent, from 0.1 to 20 weight percent, from 0.1 to 10 weight percent, from 0.5 to 60 weight percent, from 0.5 to 40 weight percent, from 0.5 to 30 weight percent, from 0.5 to 20 weight percent, from 0.5 to 10 weight percent, from 1 to 70 weight percent, from 1 to 60 weight percent, from 1 to 50 weight percent, from 1 to 40 weight percent, from 1 to 30 weight percent, from 1 to 20 weight percent, from 1 to 10 weight percent, from 10 to 70 weight percent, from 10 to 60 weight percent, from 10 to 50 weight percent, from 10 to 40 weight percent, from 10 to 30 weight percent, from 10 to 20 weight percent, from 20 to 70 weight percent, from 20 to 60 weight percent, from 20 to 50 weight percent, from 20 to 40 weight percent, from 20 to 30 weight percent, from 30 to 70 weight percent, from 30 to 60 weight percent, from 30 to 50 weight percent, from 30 to 40 weight percent, from 40 to 70 weight percent, from 40 to 60 weight percent, from 40 to 50 weight percent, from 50 to 70 weight percent, from 50 to 60 weight percent, or from 60 to 70 weight percent. In embodiments, water and alcoholic solvents may be present in the corrosion inhibitor formulation at a concentration of from 5 to 99.9 weight percent, from 5 to 90 weight percent, from 5 to 80 weight percent, from 5 to 70 weight percent, from 5 to 60 weight percent, from 5 to 50 weight percent, from 5 to 40 weight percent, from 40 to 99.9 weight percent, from 40 to 90 weight percent, from 40 to 80 weight percent, from 40 to 70 weight percent, from 40 to 60 weight percent, from 40 to 50 weight percent, from 50 to 90 weight percent, from 50 to 80 weight percent, from 50 to 70 weight percent, from 50 to 60 weight percent, from 60 to 90 weight percent, from 60 to 80 weight percent, from 60 to 70 weight percent, from 70 to 90 weight percent, from 70 to 80 weight percent, or from 80 to 90 weight percent.

In some embodiments, the corrosion inhibitor formulation includes a synergist. The synergist may act to facilitate the adsorption of an active component comprising a quaternary ammonium or pyridinium substituent onto the surface of a metal. Without being bound by theory, it is believed that the synergist adsorbs onto a metal surface while also attracting an active component comprising a quaternary ammonium or pyridinium substituent. In some embodiments, the synergist comprises a thiol substituent. In other embodiments, the synergist is an alkyl iodide. According to one or more embodiments, the synergist is thioglycolic acid, 2-mercaptoethanol, or a combination thereof. In embodiments, the synergist is present in the corrosion inhibitor formulation at a concentration of from 0.1 to 20 weight percent, from 0.2 to 20 weight percent, from 0.3 to 20 weight percent, from 0.5 to 20 weight percent, from 1 to 20 weight percent, from 1 to 15 weight percent, from 1 to 10 weight percent, from 2 to 10 weight percent, from 2 to 8 weight percent, or from 2 to 6 weight percent.

In embodiments, the corrosion inhibitor formulation includes a secondary solvent. The secondary solvent may act to clean the metal for adsorption of an active component. In embodiments, the secondary solvent comprises ethylene glycol, ethylene diamine, or combinations thereof. According to one or more embodiments, the secondary solvent is present in the corrosion inhibitor formulation at a concentration of from 0.1 to 20 weight percent, from 0.2 to 20 weight percent, from 0.3 to 20 weight percent, from 0.5 to 20 weight percent, from 1 to 20 weight percent, from 1 to 15 weight percent, from 1 to 10 weight percent, from 1 to 8 weight percent, from 1 to 6 weight percent, or from 2 to 5 weight percent.

According to one or more embodiments, the corrosion inhibitor formulation may include a surfactant. In embodiments, the surfactant may comprise an ethoxylated alcohol. According to one or more embodiments, the ethoxylated alcohol may comprise a carbon chain length of from C₅ to C₂₀, from C₈ to C₁₈, from C₁₀ to C₁₅, or from C₁₂ to C₁₄. According to one or more embodiments, the surfactant may be present in the corrosion inhibitor formulation at a concentration of from 0.1 to 10 weight percent, from 0.1 to 5 weight percent, from 0.1 to 2 weight percent, from 0.2 to 2 weight percent, from 0.2 to 1.5 weight percent, from 0.2 to 1 weight percent, from 0.2 to 0.8 weight percent, from 0.2 to 0.6 weight percent, from 0.3 to 0.6 weight percent, or from 0.4 to 0.6 weight percent.

In embodiments, the corrosion inhibitor formulation may comprise a coupling agent. The coupling agent may act to mitigate phase separation of components in the corrosion inhibitor formulation. The risk of phase separation may be especially acute in environments with a large temperature range. According to one or more embodiments, the coupling agent may be an alkyl imino dipropionic acid sodium salt. In embodiments, the coupling agent may be present in the corrosion inhibitor formulation at a concentration of from 0.1 to 10 weight percent, from 0.1 to 5 weight percent, from 0.2 to 5 weight percent, from 0.3 to 5 weight percent, from 0.3 to 3 weight percent, from 0.3 to 2 weight percent, from 0.5 to 2 weight percent, from 0.5 to 1.5 weight percent, from 0.7 to 1.5 weight percent, or from 0.8 to 1.2 weight percent.

In some embodiments, the corrosion inhibitor formulation may comprise an ethoxylated amine. The ethoxylated amine may facilitate film formation and neutralize acids present in the environment. In embodiments, the ethoxylated amine may be present in the corrosion inhibitor formulation at a concentration of from 0.1 to 10 weight percent, from 0.1 to 5 weight percent, from 0.2 to 5 weight percent, from 0.3 to 5 weight percent, from 0.3 to 3 weight percent, from 0.3 to 2 weight percent, from 0.5 to 2 weight percent, from 0.5 to 1.5 weight percent, from 0.7 to 1.5 weight percent, or from 0.8 to 1.2 weight percent.

According to one or more embodiments, the corrosion inhibitor formulation may include a supporting component. The supporting component may act cooperatively, along with the active component, to mitigate corrosion. In some embodiments, the supporting component mitigates sweet corrosion in wet environments containing both CO₂ and H₂S. According to one or more embodiments, the supporting component may comprise imidazoline. In embodiments, imidazoline may be present in the corrosion inhibitor formulation at a concentration of from 0.1 to 20 weight percent, from 0.2 to 20 weight percent, from 0.3 to 20 weight percent, from 0.5 to 20 weight percent, from 1 to 20 weight percent, from 1 to 15 weight percent, from 1 to 10 weight percent, from 2 to 10 weight percent, from 2 to 8 weight percent, or from 2 to 6 weight percent.

In embodiments, a process for inhibiting corrosion may comprise contacting a metallic surface with a corrosion inhibitor formulation according to embodiments described herein. According to embodiments, the metallic surface comprises steel. In some embodiments, the metallic surface comprises carbon steel. In embodiments, the metallic surface may be contacted with a solution containing the corrosion inhibitor formulation at a concentration by weight of from 0.5 parts per million to 500 parts per million, from 0.5 parts per million to 200 parts per million, from 0.5 parts per million to 100 parts per million, from 0.5 parts per million to 50 parts per million, from 1 part per million to 50 parts per million, from 1 part per million to 40 parts per million, from 2 part per million to 40 parts per million, from 2 part per million to 30 parts per million, from 3 part per million to 30 parts per million, from 3 part per million to 20 parts per million, from 5 part per million to 20 parts per million, or from 5 part per million to 15 parts per million. In embodiments, the metallic surface may be contacted with the corrosion inhibitor formulation at least one time, at least two times, at least three times, at least four times, or even at least five times. It should be understood that when the metallic surface is contacted with the corrosion inhibitor formulation multiple times, each contacting may be accomplished using the same or different concentrations of the compound described herein.

Many oil and gas processing facilities such as gas oil separation plants and pipelines include metallic surfaces exposed to sour well fluids. Thus, adding the corrosion inhibitor formulation described herein to the sour well fluids may mitigate corrosion of the metallic surfaces. Thus, according to one or more embodiments, a process for inhibiting corrosion includes adding the corrosion inhibitor formulation described herein to a well fluid to effect a concentration by weight of the formulation in the well fluid of from 0.5 parts per million to 500 parts per million, from 0.5 parts per million to 200 parts per million, from 0.5 parts per million to 100 parts per million, from 0.5 parts per million to 50 parts per million, from 1 part per million to 50 parts per million, from 1 part per million to 40 parts per million, from 2 part per million to 40 parts per million, from 2 part per million to 30 parts per million, from 3 part per million to 30 parts per million, from 3 part per million to 20 parts per million, from 5 part per million to 20 parts per million, or from 5 part per million to 15 parts per million.

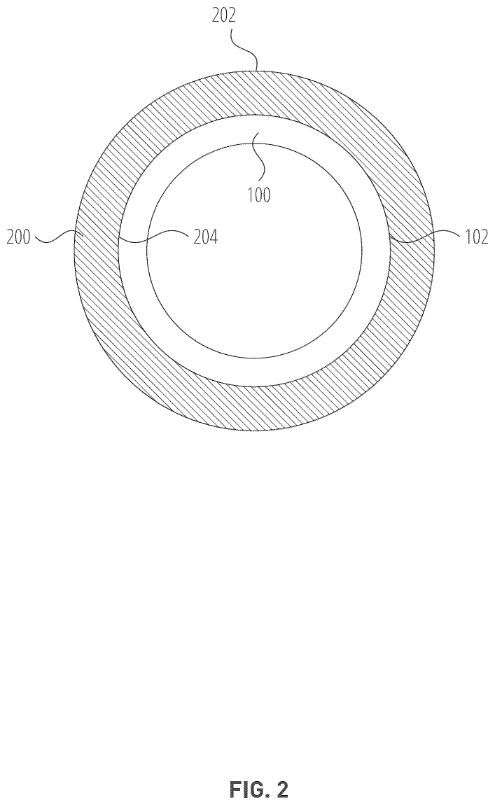

Referring now to FIG. 2, according to one or more embodiments, the corrosion-resistant substrates 150 may comprise a substrate 200 that may comprise at least a first surface 204. The term “substrate” may refer to any object with at least one surface where a solution may contact and form a film that remains on at least a portion of that surface. The corrosion-resistant substrates 150 may also comprise a corrosion-resistant film 100 that comprises at least a first surface 102 and a second surface 104 opposite the first surface 102. The corrosion-resistant film 100 may be positioned on at least a portion of the first surface 204 of the substrate 200. As depicted, the corrosion-resistant substrates 150 may have the first surface 102 of the corrosion-resistant film 100 positioned on and in direct contact with at least a portion of the first surface 204 of the substrate 200. The second surface 104 of the corrosion-resistant film 100 may be an “air-side” surface defining the outer edge of the corrosion-resistant substrate 150.

In one or more embodiments, the corrosion-resistant film 100 may be a solid. The term “solid” may refer to a material that is generally firm, stable in shape, and is not a liquid or a fluid. Accordingly, when the corrosion-resistant film 100 is a solid, the first surface 102 of the corrosion-resistant film 100 adheres to the first surface 204 of the substrate 200 so that the corrosion-resistant film 100 remains on the substrate 200 and holds its shape while the substrate 200 and/or the corrosion-resistant film 100 is moved.

In one or more embodiments, the corrosion-resistant film 100 has a thickness of from 0.1 nm to 1,000 nm, such as a thickness of from 0.1 nm to 900 nm, from 0.1 nm to 800 nm, from 0.1 nm to 700 nm, from 0.1 nm to 600 nm, from 0.1 nm to 500 nm, from 0.1 nm to 400 nm, from 0.1 nm to 300 nm, from 0.1 nm to 200 nm, from 0.1 nm to 100 nm, from 1 nm to 1,000 nm, from 10 nm to 1,000 nm, from 50 nm to 1,000 nm, from 100 nm to 1,000 nm, from 200 nm to 1,000 nm, from 300 nm to 1,000 nm, from 400 nm to 1,000 nm, from 500 nm to 1,000 nm, from 600 nm to 1,000 nm, from 700 nm to 1,000 nm, from 800 nm to 1,000 nm, from 900 nm to 1,000 nm, from 10 nm to 900 nm, from 100 nm to 800 nm, from 200 nm to 700 nm, or from 300 nm to 600 nm.

Now, referring to FIG. 2, in one or more embodiments, the substrate 200 of the corrosion-resistant substrates may be a metal pipe that comprises at least a first surface 204 and a second surface 202. The term “pipe” may refer to a tubular hollow cylinder having a circular, or near circular, cross section that is used to transport substances (for example liquids, gases, slurries, powders, small solids, etc.). The metal pipe may comprise one or more metals and one or more surfaces of the metal pipe may comprise metal oxides. For example, the metal pipe may comprise carbon steel. In some embodiments, the first surface 204 of the metal pipe may be the internal surface of the metal pipe, and the pipe may further comprise an outer surface 202. The term “internal surface” may refer to the surface of the inside of the metal pipe that is enclosed within the tubular cylinder of the metal pipe. For example, when the substrate 200 is a metal pipe and the first surface 204 is the internal surface of the metal pipe, the first surface 102 of the corrosion-resistant film 100 may be in direct contact with the internal surface of the metal pipe. Without being bound by a theory, it is believed that the corrosion-resistant film 100 being in direct contact with a least a portion of the internal surface of the metal pipe creates a barrier between the substances that flow through the metal pipe and the internal surface of the metal pipe.

The present disclosure is also directed to methods of producing corrosion-resistant substrates 150 and various embodiments of corrosion inhibitor solutions. The methods of producing corrosion-resistant substrates 150 may comprise contacting at least a portion of a first surface 204 of a substrate 200 with a corrosion inhibitor solution, where the corrosion inhibitor solution comprises a solvent and one or more bis-pyridinium compounds. Then, the methods may further comprise drying the corrosion inhibitor solution to produce the corrosion-resistant film 100 on the substrate 200, where at least a portion of the solvent is expelled from the corrosion inhibitor solution during the drying to form the solid corrosion-resistant film 100. For example, when the substrate 200 is a pipe and the first surface 204 is the internal surface of the pipe, the corrosion inhibitor solution is adhered on the internal surface of the pipe and the corrosion inhibitor solution dries on the internal surface of the pipe to form the solid corrosion-resistant film 100 on the internal surface of the pipe.

In one or more embodiments, the corrosion inhibitor solution may comprise a solvent and a bis-pyridinium compound, as described herein. In some embodiments, the solvent may comprise water, an alcohol, aromatic naphtha, or combinations thereof.

According to one or more embodiments, the corrosion inhibitor solution may comprise from 1 wt. % to 50 wt. % of the bis-pyridinium compounds disclosed and described herein. In some embodiments, the corrosion inhibitor solution may comprise from 1 wt. % to 45 wt. %, from 5 wt. % to 40 wt. %, from 10 wt. % to 35 wt. %, from 15 wt. % to 30 wt. %, or from 15 wt. % to 25 wt. % of the bis-pyridinium compounds. In some embodiments, the corrosion inhibitor solution may comprise from 5 wt. % to 50 wt. %, from 5 wt. % to 40 wt. %, from 5 wt. % to 35 wt. %, from 5 wt. % to 30 wt. %, from 5 wt. % to 25 wt. %, from 5 wt. % to 20 wt. %, from 5 wt. % to 15 wt. %, or from 5 wt. % to 10 wt. %, of the bis-pyridinium compounds.

In some embodiments, drying the corrosion inhibitor solution in order to produce the solid corrosion-resistant film 100 may include passively drying the corrosion inhibitor solution. Passively drying the corrosion inhibitor solution may refer to allowing the corrosion inhibitor solution to dry on the first surface 204 of the substrate 200 without the use of an external heat source. For example, the corrosion inhibitor solution may be allowed to dry at room temperature after contacting the first surface 204 of the substrate 200 or any similar method where the corrosion inhibitor solution is not heated with an external heat source. Further, drying the corrosion inhibitor solution in order to produce the solid corrosion-resistant film 100 may include heating the corrosion inhibitor solution with a heat source. Heating the corrosion inhibitor solution with a heat source may refer to any method where heat from an outside source is transferred to the corrosion inhibitor solution and the first surface 204 of the substrate 200 in order to dry the corrosion inhibitor solution. For example, a heat lamp may be directed onto the corrosion inhibitor solution and the first surface 204 of the substrate 200 in order to accelerate the drying time of the corrosion inhibitor solution. In another example, external processing units, liquids, or gases may transfer heat to the corrosion inhibitor solution and the first surface 204 of the substrate 200 in order to accelerate the drying time of the corrosion inhibitor solution. In some embodiments, the drying of the corrosion inhibitor solution in order to produce the solid corrosion-resistant film 100 may include both passively drying the corrosion inhibitor solution and heating the corrosion inhibitor solution with a heat source.

According to one or more additional embodiments of the present disclosure, subsurface formations may be acidized, where the bis-pyridinium compounds may be utilized to reduce corrosion of downhole metal surfaces, such as steel tubing.

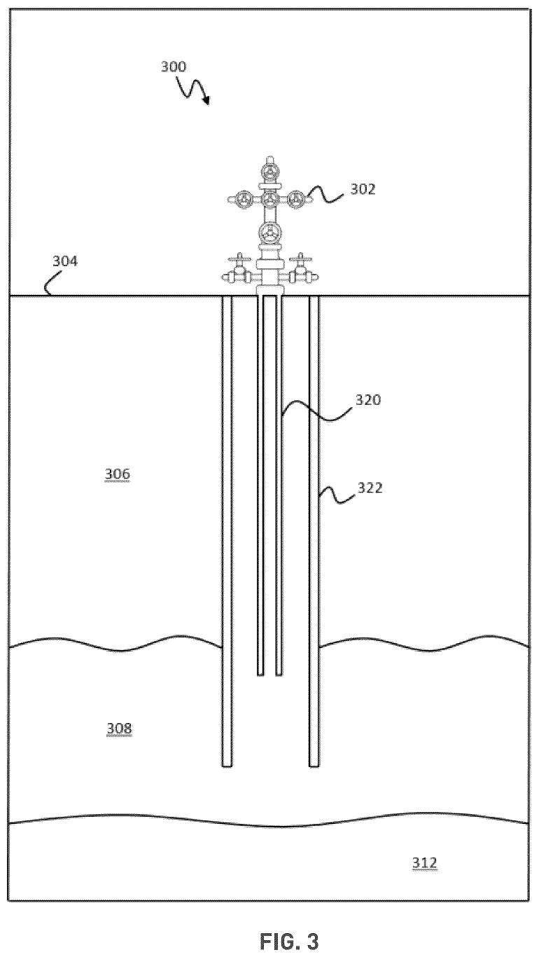

Referring now to FIG. 3, a hydrocarbon extraction well 300 is schematically depicted, where the downhole region is a cross-sectional view through a diameter of the casting 322 and tubing 320. It should be understood that FIG. 3 is a simplified depiction of a hydrocarbon extraction well 300, and other non-depicted components on a hydrocarbon extraction well 300 may be present in real-world operation. The hydrocarbon extraction well 300 may extend from the surface 304, through a subterranean zone 306, and into a subsurface formation 308 that contains extractable hydrocarbons such as oil and/or gas. Subsurface zone 312 represents another subsurface formation that does not necessarily include extractable hydrocarbons. In general, the casing 322 may form a passage to the subsurface formation 308, where cementing may be present between the natural formation and the casing 322 (not shown in FIG. 3). A wellhead 302 may connect to tubing 320 that passes through the interior of the casing 322. In general, during hydrocarbon extraction, the hydrocarbons may flow upwards through the tubing 320 and to the wellhead 302. The tubing 320 may be metal in material, such as steel. In some embodiments, the tubing 320 may be carbon steel 320.

According to embodiments, acidizing treatments may be employed as a stimulation process intended to enlarge the natural pores in material such as rock in the subsurface formation 308 and, thus, facilitate the flow of hydrocarbons that results in an increased productivity for oil and/or gas producing facilities. In general, during acidizing treatments, an acidic treatment fluid may be injected into the subsurface formation 308 from the surface 304 through the tubing 320. The acidic treatment fluid used for such application may be highly acidic and may generally cause the tubing 320 through which it passes to corrode. As such, at least a portion of the tubing 322 (such as the interior surface) is exposed to an acidic environment during acidizing treatments.

According to one or more embodiments, the acidic treatment fluid may comprise one or more acids. A wide range of acids are contemplated as suitable in the processes described herein, and an acid may be selected based on the type of rock that is to be treated and/or the intended goals of the acidizing treatment. Without limitation, according to some embodiments, the one or more acids in the acid treatment fluid may be chosen from hydrochloric acid, formic acid, acetic acid hydrofluoric acid, nitric acid, sulfuric acid, hydrobromic acid, hydroiodic acid, perchloric acid, or chloric acid.

According to embodiments, the environment in which the tubing 322 experiences is acidic and may be extremely acidic. For example, according to one or more embodiments, the acidic environment in contact with the tubing 322 may have a pH of 6 or less. In additional embodiments, the pH of the acidic environment may be less than or equal to 5.5, less than or equal to 5, less than or equal to 4.5, less than or equal to 4, less than or equal to 3.5, less than or equal to 3, less than or equal to 2.5, or even less than or equal to 2. Generally, the composition of the acidic treatment fluid and the amount used for acidizing treatment may determine the downhole acidity.

In general, the downhole conditions of the acidic environment that is contacting at least a portion of the tubing 322 may be relatively high temperature and/or relatively high pressure (sometimes referred to in industry as “HTHP”). For example, the acidic environment may have a temperature greater than or equal to 50° C., greater than or equal to 100° C., greater than or equal to 150° C., greater than or equal to 200° C., greater than or equal to 250° C., or even greater than or equal to 300° C., in some embodiments less than or equal to 1000° C. The pressure of the acidic environment may be greater than or equal to 250 psi, greater than or equal to 500 psi, greater than or equal to 750 psi, greater than or equal to 1000 psi, greater than or equal to 2000 psi, greater than or equal to 3000 psi, greater than or equal to 4000 psi, greater than or equal to 5000 psi, greater than or equal to 7500 psi, or even greater than or equal to 10,000 psi, in some embodiments less than or equal to 30,000 psi. These HTHP conditions may degrade conventional corrosion inhibitors. However, without being bound by theory, it is believed that the presently disclosed bis-pyridinium compounds may not degrade or may degrade to a lesser degree at such temperatures and pressures as compared to conventional corrosion inhibitors.

According to embodiments, during acidizing, at least a portion of the tubing 322 may be contacted by one or more bis-pyridinium compounds. In some embodiments, the bis-pyridinium compounds may be mixed into the acidic treatment fluid (or be a portion of the acidic treatment fluid). In other embodiments, the bis-pyridinium compounds may be present in a solvent or carrier fluid and be injected before, after, or concurrently with the acidic treatment fluid.

Without being bound by theory, in some embodiments, it is believed that the bis-pyridinium compound may form a film on the surface of the tubing 322, which may prevent corrosion. In other embodiments, a measurable film layer may not be formed, but corrosion on the tubing may nonetheless be mitigated. The amount of bis-pyridinium compound utilized may be sufficient to mitigate corrosion, but is not particularly limiting in the embodiments described herein. For example, from several ppm (e.g., at least 2 ppm, at least 5 ppm, at least 25 ppm, at least 50 ppm, at least 100 ppm, at least 500 ppm, or even at least 1000 ppm) of bis-pyridinium compound up to at least 1 wt. %, at least 5 wt. %, or even at least 10 wt. % of the bis-pyridinium compound may be present in the acidic environment. In some embodiments, the amount of bis-pyridinium compound may depend on the strength of the acid.

### Examples

Examples are provided herein which may disclose one or more embodiments of the present disclosure. However, the Examples should not be viewed as limiting on the claimed embodiments hereinafter provided.

### Materials and Methods

Pyridine, 1,2,7,8-diepoxyoctane, HCl (37%) and 1,4-butanediol diglycidyl ether were purchased from Sigma-Aldrich. The solvents such as diethyl ether, methanol, ethanol, and monoethyelene glycol (MEG) were also purchased from Sigma-Aldrich.

### Nuclear Magnetic Resonance (NMR) Spectroscopy

The synthesized molecules were characterized using ¹H and ¹³C {¹H} NMR spectroscopy. A JEOL 500 MHz NMR spectrometer was utilized to obtain spectra using appropriate acquisition parameters. The ¹H and ¹³C {¹H} analyses were carried in deuterated DMSO (DMSO-d₆), and the chemical shifts were set to those of the residual signal of the solvent (2.5 ppm and 39.52 ppm, respectively). The ¹H NMR spectra confirmed the structures of both bis-pyridinium compounds, Example 1 and Example 2, disclosed and described herein.

### Mass Analysis

The mass spectrum of the synthesized molecules were obtained using an Agilent 6230 time-of-flight (TOF) system equipped with electrospray ion source (ESI) and operated in positive mode. Samples were serially diluted to a final concentration of 1 ppm and were infused by a syringe at a flow rate 2 μL/min. Drying temperature was set to 200° C. The mass spectra were acquired in the 70-2200 amu mass range. The mass calibration was checked with an electrospray tune mix diluted in 1:100 methanol.

### Example 1—Synthesis of Compound of Chemical Structure #2-1

Solutions of pyridine (2 mol), 1,2,7,8-diepoxyoctane (1 mol), and HCl (37%) in a closed vessel were heated at 90° C. for 24 hours. At the end of this elapsed time, the reaction product was repeatedly washed with diethyl and freeze-dried to remove the water. The final product was recrystallized in an alcohol mixture containing methanol and ethanol in a 1:1 ratio to obtain a white solid bis-pyridinium compound. The synthesis reaction and the final compound, Example 1, are shown below.

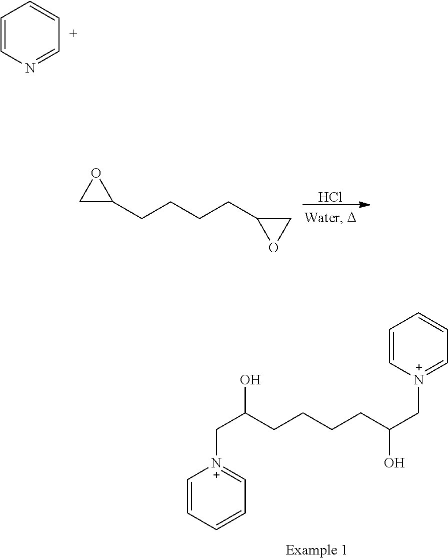 [SMILES: c1ccncc1 ; C(CCC1CO1)CC1CO1 ; Cl ; OC(CCCCC(O)C[n+]1ccccc1)C[n+]1ccccc1]

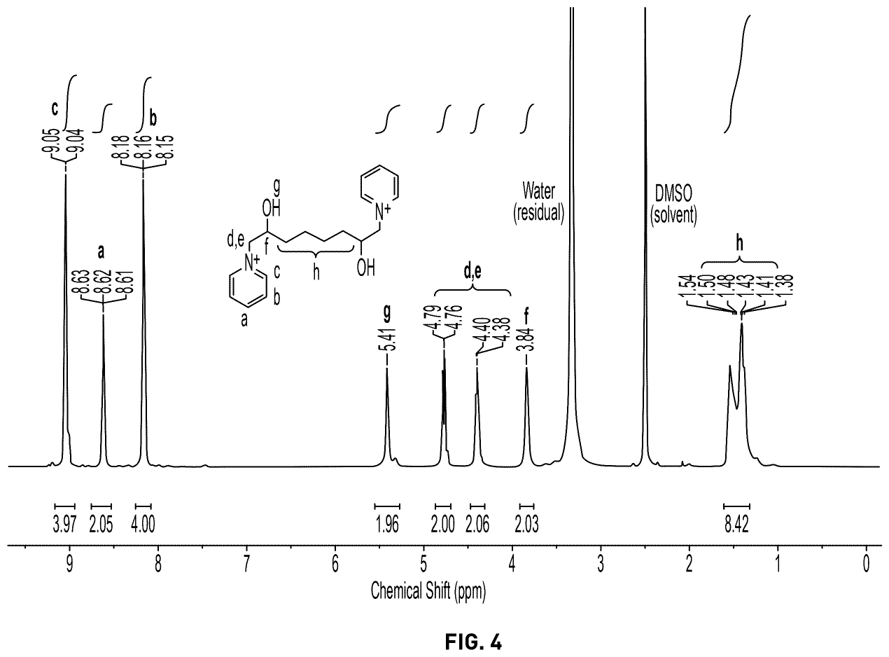

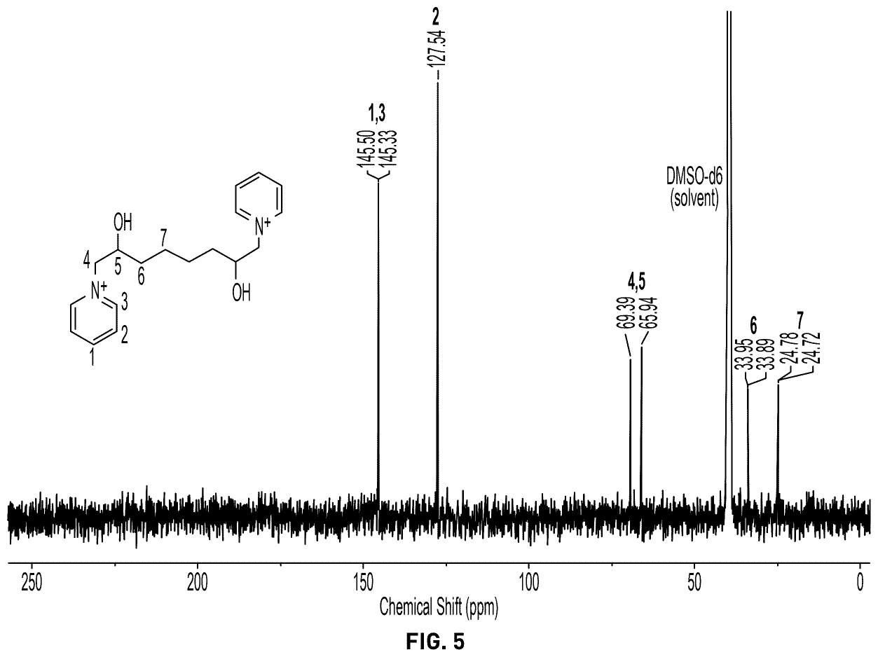

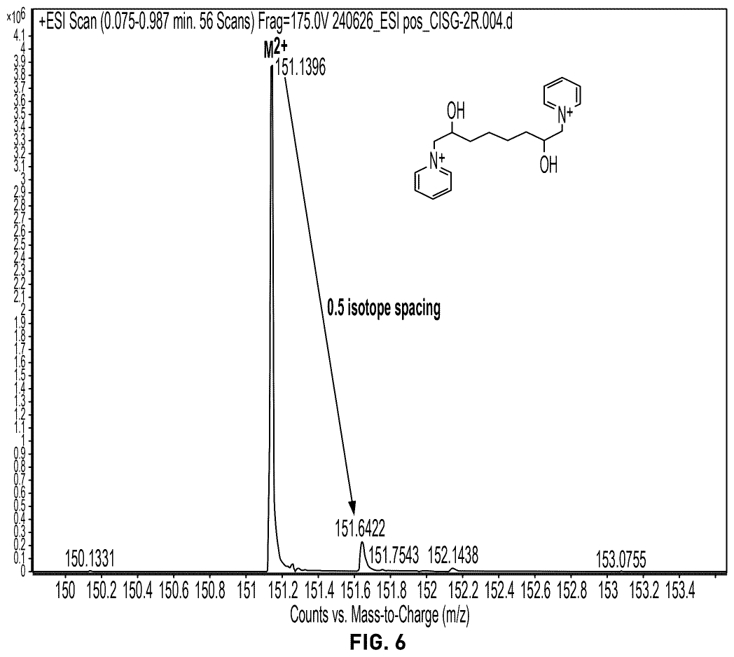

FIG. 4 provides the ¹H-NMR spectrum and FIG. 5 provides the ¹³C {¹H}-NMR spectrum of Example 1. The ¹H NMR and ¹³C {¹H} NMR spectra confirmed the structure of Example 1. FIG. 6 provides the mass spectrum of Example 1. The mass spectrum confirmed the molecular mass of Example 1.

### Example 2—Synthesis of Compound of Chemical Structure #3-1

Solutions of pyridine (2 mol), 1,4-butanediol diglycidyl ether (1 mol), and HCl (37%) in a closed vessel were heated at 90° C. for 24 hours. At the end of this elapsed time, the reaction product was repeatedly washed with diethyl and freeze-dried to remove the water. The final product was recrystallized in an alcohol mixture containing methanol and ethanol in a 1:1 ratio to obtain a white solid bis-pyridinium compound. The synthesis reaction and the final compound, Example 2, are shown below.

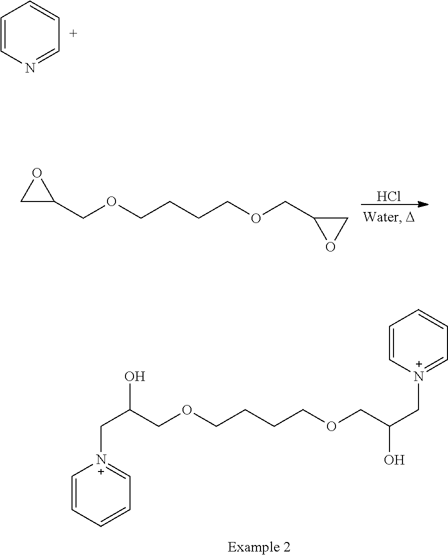 [SMILES: c1ccncc1 ; C(CCOCC1CO1)COCC1CO1 ; Cl ; OC(COCCCCOCC(O)C[n+]1ccccc1)C[n+]1ccccc1]

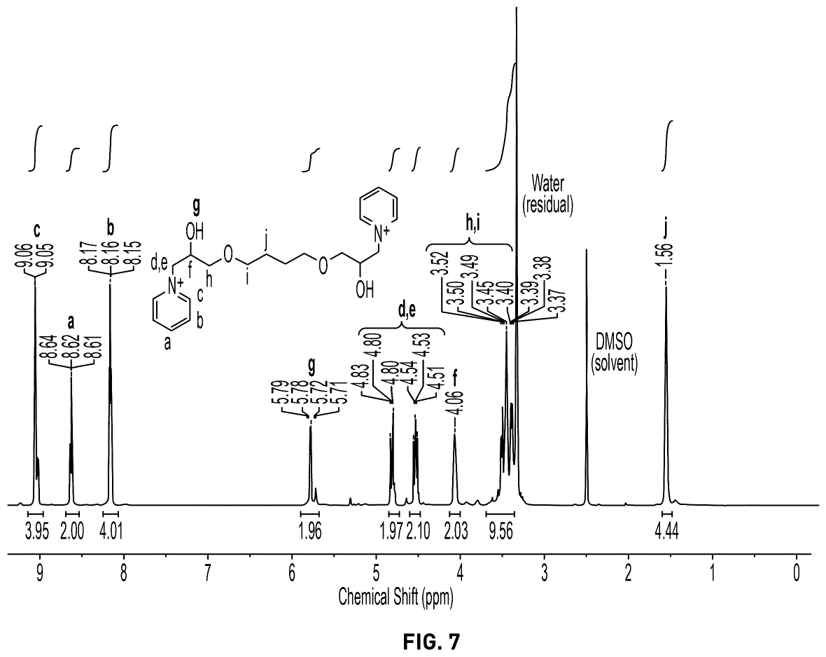

FIG. 7 provides the ¹H-NMR spectrum of Example 2. The ¹H NMR spectrum confirmed the structure of Example 2.

### Composition of Corrosion Inhibitor Formulations

The following table, Table 1, discloses a corrosion inhibitor solution that comprises Example 1 or Example 2 for performance evaluation.

TABLE 1
Performance Evaluation of Corrosion Inhibitor Formulations

| Formulation 1 | | Formulation 2 | |
|---|---|---|---|
| Components Name | Weight % | Components Name | Weight % |
| Example 1 | 30.71 | Example 2 | 50.04 |
| Monoethylene | 20.52 | Monoethylene | 33.62 |
| Glycol (MEG) | | Glycol (MEG) | |
| 2-Butoxyethanol | 4.15 | 2-Butoxyethanol | 7.09 |
| 1-Octanol | 2.78 | 1-Octanol | 4.61 |
| Methanol | 2.76 | Methanol | 4.64 |
| Water | 39.08 | | |
| Total | 100.0 | Total | 100.0 |

The performance of Formulation 1 and Formulation 2 was evaluated using a high temperature and high-pressure (HTHP) autoclave rotating cage setup and compared with a commercial corrosion inhibitor CI-2, which contains an alkyl pyridine benzyl chloride as main active component.

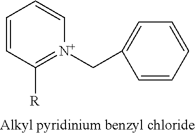

The corrosion tests were carried out in HTHP autoclave rotating cage under simulated and controlled dynamic field conditions. A four-liter reactor was constructed from C-276 alloy making it capable to withstand harsh corrosive environment. The test materials were carbon steel coupons (1018). The coupons were cleaned and degreased before and after testing following the ASTM G1 “Practice for preparing, cleaning and evaluating corrosion test specimen procedure”. The coupons were positioned in a fixed cage made out of polyether ether ketone (PEEK) material and then mounted in the autoclave. The autoclave was purged with N₂ to remove dissolved oxygen and then pressurized with the required gases (H₂S and CO₂). The autoclave was heated to the required test temperature (83° C.).

The detailed experimental conditions/parameters are given in Table 2. A mixture containing kerosene and water in a 1:1 ratio was stirred at room temperature at the presence of 10 ppm of Example 1 or Example 2. After 2 hours, kerosene was removed and the remaining water was used in the autoclave for the final test. The final pressure was maintained at the required pressure (250 psi) using high purity nitrogen gas. In all tests, the corrosion inhibitor formulations were injected immediately after fixing the coupons in the autoclave. The corrosion rate and inhibition efficiency of the samples were calculated using a weight loss by following Equation 1 and Equation 2.

$$Corrosion Rate ( C R ) ( mils per year , M P Y ) = Δ W \times 2230 \; 0 / D \times A \times T \qquad ( Equation 1 )$$

where ΔW is the weight loss of coupon in mg, D is the density of the standard mild steel coupon (C-1018) (7.89 g/cm³), A is an area of the exposed coupon (7.86 square inches), and T is the exposure time (one day). The Corrosion Rate is presented in the unit of mils per year (MPY).

$$Inhibition Efficiency ( I E \% ) = ( C {R}_{blank} - C {R}_{inhibitor} ) \times 100 / C {R}_{blank} \qquad ( Equation 2 )$$

TABLE 2
Condition for Performance Evaluation of Formula 1 and Formula 2

| | | Operational Conditions | | | | | | | Example |
|---|---|---|---|---|---|---|---|---|---|
| Water Geochemical | | | | Water | | Gas | | | Compound |
| Analysis | | Pressure | Temp. | Cut | Kerosene | Composition | Time | RPM | Concentration |
| Na⁺ | 29500 mg/L | 250 psig | 83° C. | 100 % | 0% | 7 mole % CO₂ | 24 | 400 | 20 ppm |
| Ca²⁺ | 8210 mg/L | | | | | 3 mole % H₂S | hours | | |
| Mg²⁺ | 1080 mg/L | | | | | 90 mole % N₂ | | | |
| Cl⁻ | 61800 mg/L | | | | | | | | |
| SO₄²⁻ | 1300 mg/L | | | | | | | | |
| HCO₃⁻ | 662 mg/L | | | | | | | | |
| TDS | 102552 mg/L | | | | | | | | |
| pH | 6.6 | | | | | | | | |

Table 3 provides the corrosion inhibition efficiency and corrosion rate of Formula 1 and Formula 2. As shown in Table 3, the disclosed bis-pyridinium example compounds and their corresponding formulae have a low corrosion rate and at least 20% greater corrosion inhibition efficiency compared to a commercial corrosion inhibitor, CI-2, used in gas oil separation plants (GOSPs) for a wet sour environment in the oil and gas industry.

TABLE 3
Corrosion Inhibition Efficiency and Corrosion
Rate of Formula 1 and Formula 2

| | CI-2 | Formula 1 | Formula 2 |
|---|---|---|---|
| Inhibition Efficiency (%) | 40.3 | 71.79 | 69.15 |
| Corrosion Rate (MPY) | 9.6 | 4.6 | 5 |

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

Numerous aspects of the present application are described hereinbelow as Aspects 1-20.
Aspect 1. A bis-pyridinium compound having the structure of Chemical Structure #1 or a salt thereof, wherein n is an integer greater than or equal to 0; X¹ and X² are each independently chosen from —CH₃, —NH₂, —OH, —SH, or —H; Y¹ and Y² are each independently chosen from —CH₂—, NH—, —O—, or —S—; X¹ and X² are not —H when Y¹ and Y² are CH₂—; and R^{A1}, R^{B1}, R^{C1}R^{D1}, R^{E1}, R^{A2}, R^{B2}, R^{C2}, R^{D2}, and R^{E2} are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.
Aspect 2. The bis-pyridinium compound of Aspect 1, wherein R^{A1}, R^{B1}, R^{C1}, R^{D1}. R^{E1}, R^{A2}, R^{B2}, R^{C2}, R^{D2}, and R^{E2} are each hydrogen.
Aspect 3. The bis-pyridinium compound of Aspect 1 or Aspect 2, wherein X¹ and X² are —OH.
Aspect 4. The bis-pyridinium compound of Aspect 1 or Aspect 2, wherein X¹ and X² are —NH₂.
Aspect 5. The bis-pyridinium compound of Aspect 1 or Aspect 2, wherein X¹ and X² are —SH.
Aspect 6. The bis-pyridinium compound of Aspect 1 or Aspect 2, wherein X¹ and X² are —CH₃.
Aspect 7. The bis-pyridinium compound of Aspect 1 or Aspect 2, wherein X¹ and X² are —H.
Aspect 8. The bis-pyridinium compound of any one of Aspect 1 to Aspect 6, wherein Y¹ and Y² are —CH₂—.
Aspect 9. The bis-pyridinium compound of any one of Aspect 1 to Aspect 7, wherein Y¹ and Y² are —O—.
Aspect 10. The bis-pyridinium compound of any one of Aspect 1 to Aspect 7, wherein Y¹ and Y² are —NH—.
Aspect 11. The bis-pyridinium compound of any one of Aspect 1 to Aspect 7, wherein Y¹ and Y² are —S—.
Aspect 12. The bis-pyridinium compound of any one of Aspect 1 to Aspect 11, wherein n is from 0 to 16.
Aspect 13. The bis-pyridinium compound of any one of Aspect 1 to Aspect 12, wherein n is from 0 to 8.
Aspect 14. The bis-pyridinium compound of any one of Aspect 1 to Aspect 13, wherein n equals 0; and R^{A1}, R^{B1}, R^{C1}, R^{D1}, R^{E1}, R^{A2}, R^{B2}, R^{C2}, R^{D2}, and R^{E2} are each hydrogen.
Aspect 15. The bis-pyridinium compound of any one of Aspect 1 to Aspect 13, wherein n equals 4; and R^{A1}, R^{B1}, R^{C1}, R^{D1}, R^{E1}, R^{A2}, R^{B2}, R^{C2}, R^{D2}, and R^{E2} are each hydrogen.
Aspect 16. The bis-pyridinium compound of any one of Aspect 1 to Aspect 15, wherein the compound has an average molecular mass (m/z) from 100 to 1500
Aspect 17. The bis-pyridinium compound of any one of Aspect 1 to Aspect 16, wherein the compound has an average molecular mass (m/z) from 100 to 500.
Aspect 18. The bis-pyridinium compound of any one of Aspect 1 to Aspect 17, present in an environment comprising at least 0.5 wt. % of elemental sulfur, compounds containing sulfur, or the combination thereof.
Aspect 19. A material comprising the bis-pyridinium compound of any one of Aspect 1 to Aspect 18.
Aspect 20. The material of Aspect 19, wherein the material is a well fluid comprising petroleum hydrocarbons and the bis-pyridinium compound, wherein the bis-pyridinium compound is present at a concentration by weight of 0.1 parts per million to 100 parts per million of the well fluid.
Aspect 21. The material of Aspect 19 or Aspect 20, wherein the material is a formulation for inhibiting corrosion comprising the bis-pyridinium compound; and at least one of alcoholic solvent or water, wherein the bis-pyridinium compound is present at a weight percentage from 1.0 to 60 wt. %; and the at least one of alcoholic solvent or water is present at a weight percentage from 5 to 90 wt. %.
Aspect 22. The material of any one of Aspect 19 to Aspect 21, wherein the material is a corrosion-resistant substrate comprising a substrate comprising a metallic surface; and a corrosion-resistant film positioned on at least a portion of the metallic surface of the substrate, wherein the substrate is a metal pipe; the metallic surface is an internal surface of the metal pipe; the corrosion-resistant film is solid; and the corrosion-resistant film comprises the bis-pyridinium compound.
Aspect 23. A method of acidizing a subsurface formation, the method comprising injecting an acidic treatment fluid into an subsurface formation through a tubing of an extraction well, thereby exposing at least a portion of the tubing to an acidic environment, wherein at least a portion of the tubing is contacted by the bis-pyridinium compound of any one of Aspect 1 to Aspect 18, and the acidic treatment fluid comprises one or more acids chosen from hydrochloric acid, formic acid, acetic acid hydrofluoric acid, nitric acid, sulfuric acid, hydrobromic acid, hydroiodic acid, perchloric acid, or chloric acid, or combinations thereof.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in some embodiments, the first component “consists” or “consists essentially of” that second component. It should further be understood that where a first component is described as “comprising” a second component, it is contemplated that, in some embodiments, the first component comprises at least 10%, at least 20%, at least 30%, at least 40%, at least 50%, at least 60%, at least 70%, at least 80%, at least 90%, at least 95%, or even at least 99% that second component (where % can be weight % or molar %).

It is also noted that recitations herein of “at least one” component, element, etc., should not be used to create an inference that the alternative use of the articles “a” or “an” should be limited to a single component, element, etc.

For the purposes of describing and defining the presently disclosed technology it is noted that the terms “substantially” and “about” are utilized herein to represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

What is claimed is:

1. A bis-pyridinium compound having the structure: 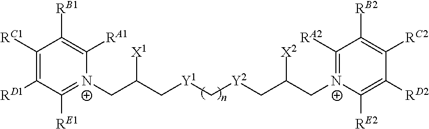
or a salt thereof, wherein:
n is an integer greater than or equal to 0;
X¹ and X² are each independently chosen from —CH₃, —NH₂, —OH, —SH, or —H;
Y¹ and Y² are each independently chosen from —CH₂—, —NH—, —O—, or —S—;
X¹ and X² are not —H when Y¹ and Y² are —CH₂—; and
R^{A1}, R^{B1}, R^{C1}, R^{D1}, R^{E1}, R^{A2}, R^{B2}, R^{C2}, R^{D2}, and R^{E2} are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

2. The bis-pyridinium compound of claim 1, wherein R^{A1}, R^{B1}, R^{C1}, R^{D1}, R^{E1}, R^{A2}, R^{B2}, R^{C2}, R^{D2}, and R^{E2} are each hydrogen.

3. The bis-pyridinium compound of claim 1, wherein X¹ and X² are —OH.

4. The bis-pyridinium compound of claim 3, wherein Y¹ and Y² are —CH₂—.

5. The bis-pyridinium compound of claim 4, wherein:
n equals 0; and
R^{A1}, R^{B1}, R^{C1}, R^{D1}, R^{E1}, R^{A2}, R^{B2}, R^{C2}, R^{D2}, and R^{E2} are each hydrogen.

6. The bis-pyridinium compound of claim 3, wherein Y¹ and Y² are —O—.

7. The bis-pyridinium compound of claim 6, wherein:
n equals 4; and
R^{A1}, R^{B1}, R^{C1}, R^{D1}, R^{E1}, R^{A2}, R^{B2}, R^{C2}, R^{D2}, and R^{E2} are each hydrogen.

8. The bis-pyridinium compound of claim 1, wherein X¹ and X² are —NH₂.

9. The bis-pyridinium compound of claim 1, wherein Y¹ and Y² are —NH—.

10. The bis-pyridinium compound of claim 1, wherein X¹ and X² are —SH.

11. The bis-pyridinium compound of claim 1, wherein Y¹ and Y² are —S— or wherein Y¹ and Y² are —CH₂—.

12. The bis-pyridinium compound of claim 1, wherein the compound has an average molecular mass (m/z) from 100 to 1500 or from 100 to 500.

13. The bis-pyridinium compound of claim 1, wherein n is from 0 to 16.

14. The bis-pyridinium compound of claim 1, wherein n is from 0 to 8.

15. The bis-pyridinium compound of claim 1, present in an environment comprising at least 0.5 wt. % of elemental sulfur, compounds containing sulfur, or the combination thereof.

16. A material comprising a bis-pyridinium compound having the structure: 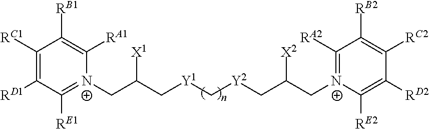
or a salt thereof, wherein:
n is an integer greater than or equal to 0;
X¹ and X² are each independently chosen from —CH₃, —NH₂, —OH, —SH, or —H;
Y¹ and Y² are each independently chosen from —CH₂—, —NH—, —O—, or —S—;
X¹ and X² are not —H when Y¹ and Y² are —CH₂—; and
R^{A1}, R^{B1}, R^{C1}, R^{D1}, R^{E1}, R^{A2}, R^{B2}, R^{C2}, R^{D2}, and R^{E2} are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

17. The material of claim 16, wherein the material is a well fluid comprising petroleum hydrocarbons and the bis-pyridinium compound, wherein the bis-pyridinium compound is present at a concentration by weight of 0.1 parts per million to 100 parts per million of the well fluid.

18. The material of claim 16, wherein the material is a formulation for inhibiting corrosion comprising:
the bis-pyridinium compound; and
at least one of alcoholic solvent or water,
wherein:
the bis-pyridinium compound is present at a weight percentage from 1.0 to 60 wt. %; and
the at least one of alcoholic solvent or water is present at a weight percentage from 5 to 90 wt. %.

19. The material of claim 16, wherein the material is a corrosion-resistant substrate comprising:
a substrate comprising a metallic surface; and
a corrosion-resistant film positioned on at least a portion of the metallic surface of the substrate,
wherein:
the substrate is a metal pipe;
the metallic surface is an internal surface of the metal pipe;
the corrosion-resistant film is solid; and
the corrosion-resistant film comprises the bis-pyridinium compound.

20. A method of acidizing a subsurface formation, the method comprising:
injecting an acidic treatment fluid into an subsurface formation through a tubing of an extraction well, thereby exposing at least a portion of the tubing to an acidic environment,
wherein:
at least a portion of the tubing is contacted by a bis-pyridinium compound having the structure: 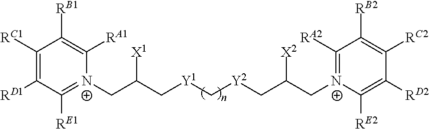
or a salt thereof, wherein:
n is an integer greater than or equal to 0;
X¹ and X² are each independently chosen from —CH₃, —NH₂, —OH, —SH, or —H;
Y¹ and Y² are each independently chosen from —CH₂—, —NH—, —O—, or —S—;
X¹ and X² are not —H when Y¹ and Y² are —CH₂—; and
R^{A1}, R^{B1}, R^{C1}, R^{D1}, R^{E1}, R^{A2}, R^{B2}, R^{C2}, R^{D2}, and R^{E2} are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group; and
the acidic treatment fluid comprises one or more acids chosen from hydrochloric acid, formic acid, acetic acid hydrofluoric acid, nitric acid, sulfuric acid, hydrobromic acid, hydroiodic acid, perchloric acid, or chloric acid, or combinations thereof.
